(19) United States
(12) Patent Application Publication
(10) Pub. No.: US 2026/0033821 A1
(43) Pub. Date: Feb. 5, 2026

(54) RETRACTOR FOR BIPORTAL ENDOSCOPIC SURGERY

(71) Applicant: JASPER MEDTECH PTY LTD., Sydney (AU)

(72) Inventor: Ralph MOBBS, Sydney (AU)

(21) Appl. No.: 19/288,232

(22) Filed: Aug. 1, 2025

(30) Foreign Application Priority Data
Aug. 2, 2024 (AU) 2024902411

(51) Int. Cl. A61B 17/02; A61B 1/00; A61B 17/00

(52) U.S. Cl. CPC A61B 17/0218; A61B 1/00147; A61B 17/0206; A61B 2017/00477; A61B 2017/00862; A61B 2017/0225; A61B 2017/0256

(57) ABSTRACT

There is provided a retractor to maintain triangulation during biportal endoscopic surgery, the retractor comprising: a head and a tail connected by an elongate member; wherein: the head is adapted to engage with a central portion adjacent the tail; a first portion of the elongate member is adapted to engage with a surgical instrument; and the retractor is adapted to traverse a working port and a viewing port such that, in use, the tail is advanced into the head to retract tissue surrounding the working port and the viewing port.

### CROSS-REFERENCE TO RELATED APPLICATION(S)

The present application claims priority from Australian Provisional Patent Application No. 2024902411 filed 2 Aug. 2024, the entire contents of which are incorporated herein by cross-reference.

### TECHNICAL FIELD

The technology relates to a retractor to maintain triangulation during biportal endoscopic surgery. In particular, the device traverses both ports and functions to retract tissue and maintain triangulation by engaging with the insertion tube of an endoscope to maintain the relative orientation of the endoscope and the working port.

### BACKGROUND

Biportal endoscopic surgery, particularly for the spine, is a relatively new technique but has become standard of care at many centers for lumbar decompressions and discectomies due to minimal muscle damage, minimal postoperative back pain and early recovery. In biportal endoscopic surgery, the separation of viewing port and working port allows greater freedom of movement for the surgical instruments as the camera and instruments move independently providing unrestricted vision. Moreover, the continuous irrigation fluid pressure decreases bleeding and helps to irrigate out debris. Hence, biportal endoscopic surgery has become quite popular and its adoption is assisted by the use of standard arthroscopic lenses, familiar view of anatomy, and use of conventional instruments.

However, the biportal endoscopic technique has a complex learning curve and a risk of higher rates of complications for less experienced surgeons. Biportal endoscopic surgery requires triangulation of a working area, for example a damaged disc, with an endoscope inserted from one side of the area for viewing and the surgical instruments from the other side for working purposes. When triangulation is established between the endoscope and instruments, it must be maintained and imprecise movement of the endoscope relative to the working port can obscure the working area necessitating repositioning of the endoscope or instruments, which may lead to unnecessary tissue damage.

Further, during surgery, instruments must traverse the working port numerous times and each traverse carries a risk of tissue damage along the port. It is therefore advantageous to create a relatively open working port and limit the number of times an instrument traverses the port.

Accordingly there is a need for improved devices and methods for biportal endoscopic surgery.

The present inventor has developed a device to traverse both ports, retract tissue and maintain the relative position of an endoscope and a working port.

### SUMMARY

In a first aspect, there is provided a retractor to maintain triangulation during biportal endoscopic surgery, the retractor comprising a head and a tail connected by an elongate member; wherein: the head is adapted to engage with a central portion adjacent the tail; a first portion of the elongate member is adapted to engage with a surgical instrument; and the retractor is adapted to traverse a working port and a viewing port such that, in use, the tail is advanced into the head to retract tissue surrounding the working port and the viewing port.

In some embodiments, the elongate member includes one or more relief zones generally defined as a pivot portion located between the first portion and the second portion of the elongate member.

In some embodiments, the elongate member comprises one or more regions adapted to bend or turn about the one or more relief zones.

In some embodiments, the one or more relief zones are integrally formed with the elongate member. In some embodiments, the one or more relief zones are provided as a pivotable structure connecting the one or more regions of the elongate member.

In some embodiments, in use, the retractor is adapted to maintain triangulation during the surgical procedure at an angle between the working port and the viewing port. For example, the angle between the working port and the viewing port may substantially correspond with an angle defined by the one or more relief zones

In some embodiments, the first portion of the elongate member comprises at least one or more flanges configured to engage with the surgical instrument. For example, the one or more flanges may laterally extend from the central portion of the elongate member to form a channel with a first longitudinal axis that substantially aligns with a longitudinal axis defined by the central portion of the elongate member.

In some embodiments, the elongate member provides one or more shaft connection means that are adapted to advantageously removably retain the surgical instrument received therein.

In some embodiments, an inner radius of the one or more shaft connection means substantially corresponds with an outer radius of the surgical instrument.

In some embodiments, the one or more shaft connection means are substantially C-shaped, U-shaped, or has a rectilinear profile.

In some embodiments, each of the one or more flanges or lateral extensions is of the same or different material as the elongate member.

In some embodiments, the surgical instrument is an endoscope or a working scope, and the first portion of the elongate member is adapted to engage with an insertion tube or external profile of the surgical instrument.

In some embodiments, in use, the channel is positioned at the working port or the viewing port, to protect the tissue surrounding the respective working port or viewing port.

In some embodiments, the retractor further comprises a flexible transition zone between the elongate member and the head, wherein the transition zone is configured to retract the tissue surrounding the surgical instrument. For example, the transition zone may be in the shape of a substantially arcuate hook defined by a bend portion and a tip portion.

In some embodiments, the head or the elongate member is configured to turn or pivot about the transition zone.

In some embodiments, the retractor further comprises a second flexible transition zone configured to retract tissue surrounding, for example, the second port.

In some embodiments, the head comprises a housing structure and one or more flange members laterally extending thereof, wherein the one or more flange members and/or a surface of the housing structure define a contact surface configured to distribute a load onto the patient's skin to thereby stabilize the retractor, in use. The one or more flange members can advantageously assist with stabilizing and maintaining triangulation of the working port and viewing for the duration of the surgical procedure. For example, the one or more flange members may be substantially coplanar or non-coplanar with a plane defined by the surface of the housing structure.

In some embodiments, the one or more flange members are substantially flat and dome-shaped.

In some embodiments, the head comprises an engagement means configured to engage with the tail and/or the elongate member. For example, the engagement means may be provided as a pawl member.

In some embodiments, the tail and/or the central portion of the elongate member comprise a plurality of teeth configured to engage with the pawl member.

In some embodiments, the pawl member may provide complementary teeth members configured to engage with the teeth of the elongate member.

In some embodiments, the engagement means further comprises a release mechanism configured to enable the engagement means to releasably engage with the tail and/or elongate member.

In a second aspect there is provided a method of maintaining triangulation during biportal endoscopic surgery, the method comprising advancing the tail of a retractor of the first aspect through both a working port and a viewing port, advancing the tail into the head of the retractor to retract tissue surrounding the working port and the viewing port; and engaging one or more portions of the elongate member of the retractor with a surgical instrument such as an endoscope, preferably an insertion tube of the endoscope

In some embodiments, the elongate member is configured to releasably retain the surgical instrument.

In some embodiments, the method further comprising: maintaining an angle between the working port and the viewing port throughout the surgical procedure, wherein the angle substantially corresponds with an angle defined by the one or more portions of the elongate member.

In some embodiments, the method further comprising: engaging the tail and/or the elongate member with the head of the retractor to thereby define a closed configuration.

In some embodiments, the head of the retractor provides a contact surface that is configured to distribute a load onto the patient's skin to thereby stabilise the retractor in use.

In some embodiments, the surgical instrument is an endoscope, preferably an insertion tube of the endoscope.

### Definitions

Throughout this specification, unless the context clearly requires otherwise, the word ‘comprise’, or variations such as ‘comprises’ or ‘comprising’, will be understood to imply the inclusion of a stated element, integer or step, or group of elements, integers or steps, but not the exclusion of any other element, integer or step, or group of elements, integers or steps.

Throughout this specification, the term ‘consisting of’ means consisting only of.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

Unless the context requires otherwise or specifically stated to the contrary, integers, steps, or elements of the technology recited herein as singular integers, steps or elements clearly encompass both singular and plural forms of the recited integers, steps or elements.

In the context of the present specification the terms ‘a’ and ‘an’ are used to refer to one or more than one (ie, at least one) of the grammatical object of the article. By way of example, reference to ‘an element’ means one element, or more than one element.

In the context of the present specification the term ‘about’ means that reference to a figure or value is not to be taken as an absolute figure or value, but includes margins of variation above or below the figure or value in line with what a skilled person would understand according to the art, including within typical margins of error or instrument limitation. In other words, use of the term ‘about’ is understood to refer to a range or approximation that a person or skilled in the art would consider to be equivalent to a recited value in the context of achieving the same function or result.

The reader will also appreciate and understand that any use of relative, directional or positional terms as described herein (e.g., upper, lower, central, side, on top etc) are used to convey and describe to the reader the relative position, direction, and/or orientation of the retractor 10 as illustrated in the accompanying drawings, as well as the components themselves. For example, when viewing FIG. 1A into the page, the upper surface 202 of the head 200 is visible to the reader, whereas the lower surface 204 of the head 200 is not. Other variations in the position or orientation of the components of the retractor 10 are contemplated within the scope of this disclosure depending on the user's specific situation.

Those skilled in the art will appreciate that the technology described herein is susceptible to variations and modifications other than those specifically described. It is to be understood that the technology includes all such variations and modifications. For the avoidance of doubt, the technology also includes all of the steps, features, and compounds referred to or indicated in this specification, individually or collectively, and any and all combinations of any two or more of said steps, features and compounds.

In order that the present technology may be more clearly understood, preferred embodiments will be described with reference to the following drawings and examples.

### BRIEF DESCRIPTION OF THE DRAWINGS

FIGS. 1A-1C illustrate a top view, a side view, and a perspective view, respectively, of a retractor being shown in an open configuration; in accordance with one embodiment.

FIG. 2 illustrates a perspective view the retractor of FIG. 1 shown in a closed configuration;

FIG. 3 illustrates a close-up cross sectional side view of a head of the retractor of FIGS. 1-2 and an engagement means engaged with one or more teeth of an elongate member of the retractor;

FIG. 4 illustrates a close-up cross sectional side view of the retractor head of FIGS. 1-3 including a release mechanism; and

FIG. 5 illustrates a perspective view of the retractor of FIG. 1-4, when in use.

### DESCRIPTION OF EMBODIMENTS

The description should be read with reference to the drawings in which similar elements in different drawings are numbered the same. The drawings, which are not necessarily to scale, depict illustrative embodiments and are not intended to limit the scope of the invention.

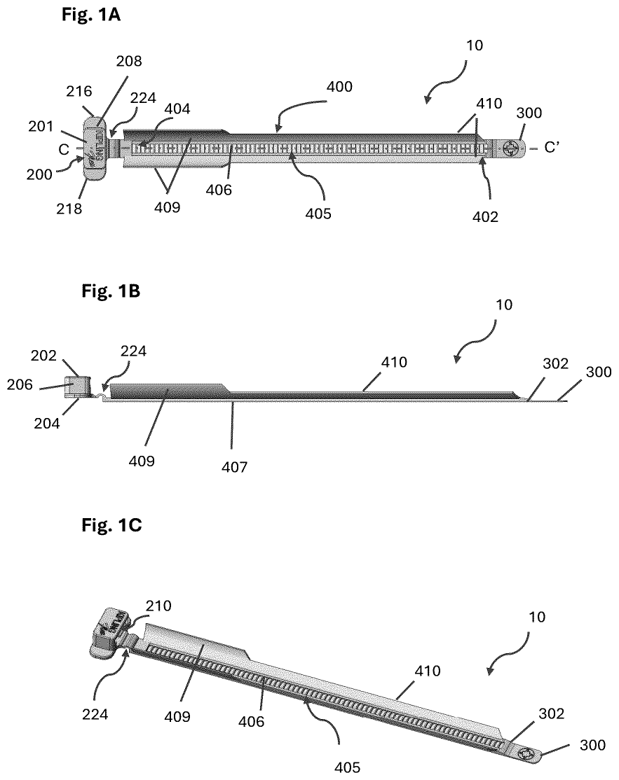

With reference to FIGS. 1-5, there is provided a retractor 10 configured to retract tissue and to maintain triangulation at a particular angle during biportal endoscopic surgery. The retractor 10 generally comprises a head 200, and a tail 300 connected by an elongate member 400 extending therebetween.

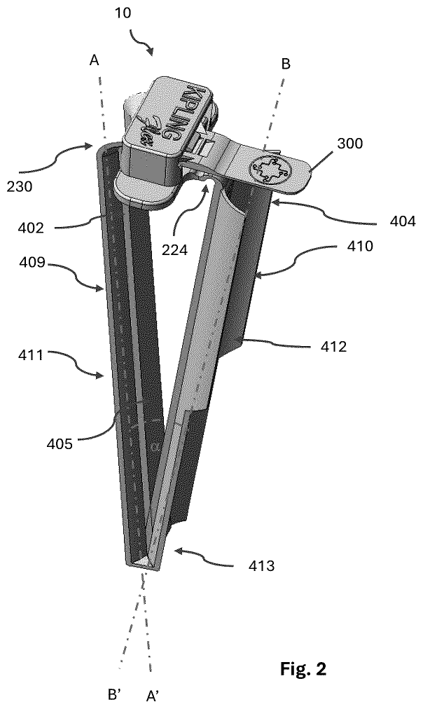

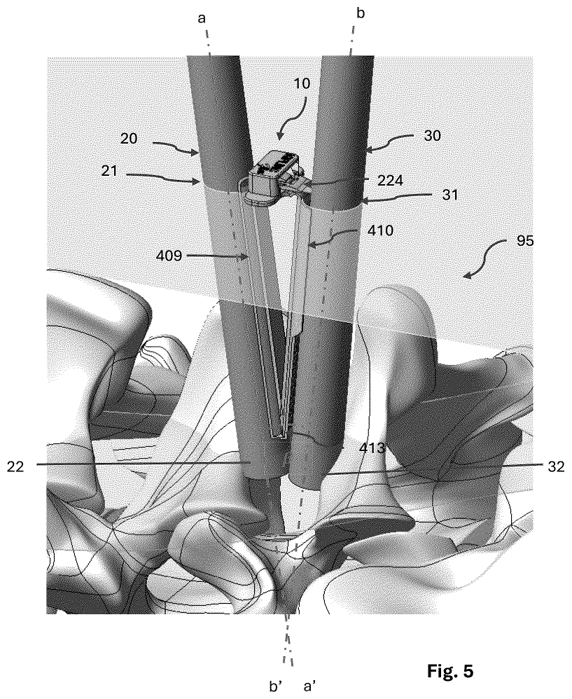

FIGS. 1A-1C illustrate an example of the retractor 10 provided in an open configuration and FIG. 2-4 illustrate examples of the retractor 10 provided in a closed configuration. FIG. 5 illustrates an example of the retractor 10 that is adapted to engage with a working port 20 and a viewing port 30. The retractor 10 is adapted to retract tissue 95, hold the ports 20,30 at a particular position/orientation relative to the patient, and to maintain triangulation of the ports 20,30 at an angle β for the duration of the surgical procedure, such as biportal endoscopic surgery. The structural components and mechanisms of the retractor 10 will now be described herein.

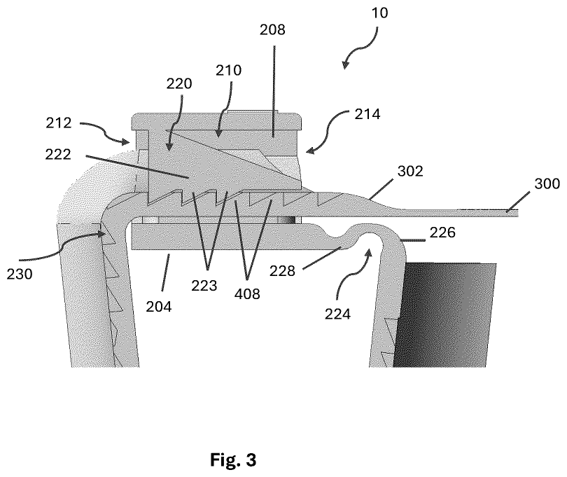

The head 200 of the retractor 10 is generally in the form of a rectangular housing structure 201 that is configured to receive the tail 300 and/or elongate member 400 therein. The housing structure 201 has an upper surface 202 and a lower surface 204 both extending between a first side wall 206 and a second side wall 208. An aperture 210 is generally defined within the housing structure 201 by the opposing side walls 206, 208 and opposing surfaces 202, 204. With reference to FIG. 3, the aperture 210 has a receiving region 212 through which the tail 300 and elongate member 400 are configured to pass through, and an exit region 214 through which the tail 300 and/or elongate member 400 are configured to protrude through.

Extending laterally from the housing structure 201 of the head 200 is a first flange member 216 and a second flange member 218. In the illustrated examples, the opposing flange members 216, 218 are shown to be in the form of substantially flat, dome-shaped members and are substantially co-planar with a plane defined by the lower surface 204 of the head 200.

With reference to FIG. 5, the opposing flange members 216 and 218 are configured to be sit or rest on top of the patient's skin 95, while the retractor 10 is in use. While in use, the flange members 216, 218 and/or the flat lower surface 204 of the head 200 generally define a contact surface area that is configured to sit on top of and rest against the patient's skin 95, to thereby distribute a contact load onto the surface of the patient's skin 95. This can advantageously assist with stabilizing and maintaining triangulation of the working port 20 and viewing port 30 at the defined angle β for at least the duration of the surgical procedure.

In some embodiments, the shape of the opposing flange members 216, 218 may be circular, triangular, or rectangular and such variations are contemplated within the scope of this disclosure. In some embodiments, the flange members 216, 218 may laterally extend from the opposing side walls 206, 208 and/or or from the upper surface 202 towards the patient's skin.

In some embodiments, the transversely extending flange members 216, 218 may be non-coplanar with the plane defined by the lower surface 204 and can define a substantially curved and/or rectilinear contact surface area to match and/or conform to the external profile of the patient's skin 95.

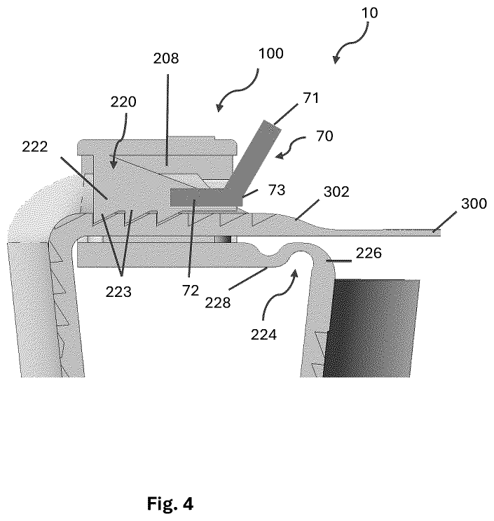

Provided within the housing structure 201 of the head 200 is an engagement means 220 that is in the form of a triangular pawl 222 (see FIGS. 3-4) and the mechanisms of the engagement means 220 will later be described herein.

As shown in the illustrated embodiment, the head 200 of the retractor 10 further includes a flexible transition zone 224 connecting the housing structure 201 of the head 200 with the elongate member 400. When viewed from a side view (such as FIGS. 3-4), the flexible transition zone 224 is shown to be in the shape of a substantially arcuate hook generally defined by a bend portion 226 connecting to the elongate member 400, and a tip portion 228 which connects to the lower surface 202 of the housing structure 201.

The head 200 or the elongate member 400 is configured to turn or pivot about the transition zone 224 to allow the head 200 to retract and/or hook the patient's skin 95 during the surgical procedure. For example, with reference to FIG. 5, in use, the transition zone 224 can advantageously be used to retract tissue surrounding the adjacent port, eg the working port 30.

In the illustrated embodiment, the housing structure 201 of the head 200 is substantially rectangular in shape. In other embodiments, the housing structure 201 may be triangular, circular or dome shaped, and such variations in profile are contemplated within the scope of this disclosure depending on how the user wishes to design the retractor 10.

The aperture 210 in the illustrated embodiment is similarly shown to be substantially rectangular shape. In other embodiments, the profile of the aperture 210 may vary, depending on the shape and/or configuration of the housing structure 201, and/or the tail 300 and elongate member 400 received therein. In some embodiments, the aperture 210 may generally be in the shape of a slot, groove, trapezoidal etc.

In some embodiments, the aperture 210 may only provide a receiving region 212, whereby the exit region 214 is sealed or enclosed by a surface of the housing structure 201 (not shown herein). For example, other embodiments may be contemplated wherein the head 200 would prevent the tail 300 and/or elongate member 400 from protruding therethrough, to keep ratchet and pawl mechanism generally within the aperture 210 of the housing structure 201 of the head 200.

In other embodiments not illustrated herein, the elongate member 400 may also provide an opposing second flexible transition zone (not illustrated but generally indicated by the reference numeral 230 in FIGS. 2 and 3). Similarly, the second transition zone 230 may also be used to advantageously retract tissue 95 surrounding the, eg, second port 20.

Extending from the elongate member 400 is a tail 300. As shown in FIGS. 1-5, the tail 300 is dimensioned to be received by the head 200 and to pass through the aperture 210 of the head 200. As better shown in FIGS. 2-5, the tail portion 300 is dimensioned to be received through the receiving region 212 of the aperture 210 and protrude through the exit region 214. The illustrated examples show the tail 300 in the shape of a substantially flat and thin dome-shaped strip that connects to the elongate member 300 via a sloped portion 302.

In other embodiments, the shape and/or configuration of the tail 300 can be adjusted according to, eg, the profile of the aperture 210. For example, the tail 300 may be thinner in one dimension and/or it may have a rounded end to facilitate entry into and to pass through the head 200.

With reference to FIGS. 1-2, the elongate member 400 is in the form of an elongate strip that longitudinally extends between the head 200 and the tail 300. The elongate member 400 provides a first portion 402 proximal the head 200, a second portion 404 proximal the tail 300, and a central longitudinal portion 405 extending therebetween with an upper surface 406 and a lower surface 407. With reference to FIGS. 1A-1C, the central portion 405 generally defines a longitudinal axis C-C′ and provides on its upper surface 406 one or more teeth 408 defined along the longitudinal axis C-C′. With reference to FIGS. 3-4, the one or more teeth 408 are dimensioned and configured to engage with the complementary teeth 223 of the pawl 222 of the engagement means 220 to define the rachet and pawl mechanism.

In the illustrated embodiment, the teeth 408 are provided along the upper surface 406 of the central portion 405 of the elongate member 400. In some embodiments (not illustrated herein), the teeth 408 can be provided on the lower surface 407 of the elongate member 400 to similarly engage with complementary teeth provided on the lower surface 204 of the housing structure 201 (not illustrated) to provide a similar ratchet and pawl or locking mechanism.

As shown in FIG. 2, the elongate member 400 provides one or more relief zones generally defined as a pivot portion 413 that is located between the first portion 402 and second portion 404 of the elongate member 400. With reference to FIG. 5, while in use, the second portion 404 of the elongate member 400 may pivot or turn about the pivot portion 413 to thereby define the acute angle β.

The pivot portion 413 can advantageously facilitate and maintain an acute bend between the first portion 402 and second portion 404 of the elongate member 400 at an acute angle α. With reference to FIG. 5, the pivot portion 413, while in use, can also facilitate and maintain an acute bend adjacent or proximal the internal ends 21, 31 of the working and viewing ports 20, 30 at an acute angle β.

In the illustrated embodiment, the pivot or rotating portion 413 is shown to be integrally formed with the elongate member 400. In other embodiments, the pivot portion 413 may be provided as a separate pivotable structure connected to the elongate member-eg, as a separate hinge component.

In some embodiments, it is envisaged that the elongate member 400 may comprise one or more additional pivot portions or joints 412 to allow the retractor 10 to articulate and to conform/engage with the external profile of the ports 20,30 and/or the skin of the patient 95, while in use.

In some forms, the pivot portion 413 may be an area of the elongate member 400 comprising a more ductile material or may be an area of reduced dimensions. Alternatively the pivot portion 413 may be a zone comprising material arranged in a wave formation (e.g. sine wave or zig zag).

The elongate member 400 further includes one or more curved flange walls 409, 410 transversely extending from the central portion 405 of the elongate member 400. In the illustrated examples, the at least one flange walls 409 proximal the head 200 and the at least one flange wall 410 proximal the tail 300 are substantially arcuate in shape and are configured to match with the external profile of the ports 20,30.

In the illustrated embodiment, the second flange walls 410 are shown to have an arcuate length greater than the first flange walls 409. In some embodiments, the arcuate length of these walls 409, 410 may vary by, eg, having a larger arcuate length which defines a deeper ‘C’ or ‘U’ shaped channel 411, 412, or a shorter arcuate length 409, 410 defining a more shallow ‘C’ or ‘U’ channel 411, 412, depending on the external diameter of respective the ports 20, 30.

In the illustrated embodiment, the flange walls 409, 410 are arcuate or curved in shape and are dimensioned to each respectively define shallow concave channels 411, 412 with a cross section that substantially aligns with the cross section or external profile of the respective surgical instruments 20, 30.

With reference to FIGS. 2 and 5, the flange walls 409, 410 and/or the central portion 405 of the elongate member 400 generally define one or more shaft connection means that is adapted to provide a ‘clip-fit’ or ‘push-fit’ mechanism that can removably retain, interlock or engage with the shaft of the respective ports 20, 30 received therein. The inside radius of the shaft connection means substantially correspond with the outside radius of the shaft of the respective instruments 20, 30. In the illustrated embodiment, the shaft connection means are substantially C-shaped or U-shaped and, in other embodiments, other curved or rectilinear profiles are contemplated within this disclosure.

Each of the channels 411, 412 have a cross section with suitable dimensions to receive the respective ports 20, 30 and space apart the sides of the working channel such that surgical instruments can traverse the working/viewing port 20, 30 with ease (as further discussed herein).

In the illustrated embodiment, the flanges 409, 410 are substantially the same dimensions on either side of the elongate member 400. Various alternate embodiments are envisaged, including flanges of different widths and lengths on either side of the elongate member 400. The flanges 409, 10 may be in the form of multiple fingers or protrusions (lateral extensions) on either side of the elongate member 400, for example to produce a scalloped edge.

In some embodiments, the flanges 406, 408 and/or the teeth 408 of the elongate member 400 are adapted to engage with the surgical instruments 20, 30. For example, with reference to FIG. 5, the first flange portion 409 that extends along the first portion 402 of the elongate 400 is adapted to engage with a first surgical instrument 20 (such as a working port) and the second flange portion 410, which extends along the second portion 404 of the elongate 400, is adapted to engage with a second surgical instrument 30 (such as a viewing port).

In some embodiments, the material of the shaft connection means or the flange walls 409, 410 can be manufactured from a rigid but partially flexible material (eg plastic) to facilitate the removable retaining mechanism.

In some embodiments, the arcuate length of the flange walls 409, 410 may be the same depending on the size and/or external profile of the respective surgical ports 20, 30 that the flange walls 409, 410 are configured to engage therewith.

In some forms, the arcuate length of the flange walls 409, 410 may vary and may not be constant and these variations in lengths and/or shapes are considered within this disclosure.

The retractor 10 in its closed configuration will now be discussed with particular reference to FIGS. 2-5. The illustrated embodiment show the shaft connecting means of the first portion 402 of the elongate member 400 receiving the working port 20, and the shaft connecting means of the second portion 404 receiving the viewing port 30.

In other embodiments, the configuration between the first portion 402 and second portion 404 with the respective ports 20 and 30 may interchange or may include other types of surgical equipment depending on the preferred set up of the surgical procedure.

The second portion 404 of the elongate member 400 is bent or pivoted about a rotational axis defined by the pivot portion 412. With reference to FIGS. 1-2, the first concave channel 411 defines a longitudinal axis A-A′ that substantially aligns with the longitudinal axis C-C′ of the central portion 405 of the elongate member 400. Similarly, the second concave channel 412 defines a longitudinal axis B-B′ that substantially aligns with the longitudinal axis C-C′ of the central portion 405.

With reference to FIG. 5, in use, the retractor 10 is configured to triangulate and maintain the surgical instruments 20, 30 at the angle β for the duration of the surgical procedure. With the retractor 10 engaged with the surgical instruments 20 and 30 (eg, via the shaft connection means discussed previously), the axis A-A′ of the first concave channel 411 is configured to co-axially align with the axis a-a′ of the working port 20, and the axis B-B′ of the second concave channel 412 is configured to co-axially align with the axis b-b′ of the viewing port 30.

This configuration of the retractor 10 advantageously triangulates and maintains the working scopes 20, 30 at the angle β for the duration of the surgical procedure. This fixed triangulation can advantageously minimize unwanted lateral movements from the working scope 20, 30 that may harm the patient at the particular site. This may involve tissue damage due to the insertion, removal and/or movement of the ports 20,30 and/or other surgical instruments used within the ports 20, 30 during the surgical procedure.

In some embodiments, the angle α defined between the first and second channel 411, 412 of the elongate member 400 would substantially correspond with the angle β between the working port and viewing port 20,30.

The ratchet and pawl or locking mechanism 220 will now be described with particular reference to FIGS. 3 and 4. In some embodiments, the engagement means 220 includes a triangular pawl member 222 with one or more complementary teeth 223. In the closed configuration, the one or more teeth 408 of the elongate member 400 are dimensioned to engage with the one or more complementary teeth 223 of the pawl 222 such that, as the tail 300 is advanced through the head 200, the teeth 408 move past the complementary teeth 223 of the pawl 222 which together form a ratchet and pawl mechanism. The one or more teeth 408 of the elongate member 400 and the complementary teeth 223 of the pawl 222 would then interlock/inter-engage to prevent retreat of the tail 300 and secure the elongate member 400 relative to the head 200.

In some embodiments, the locking mechanism may be defined by one or more teeth 408 provided on the lower surface 407 of the elongate member 400 configured to engage with complementary teeth 223 of a pawl 222 extending from the lower surface 204 of the housing structure 201.

With reference to FIG. 4, the engagement means 220 may optionally include a release mechanism 70. In the illustrated example, the release mechanism 70 is the form of a an ‘L-shaped’ lever 70 with a distally located handle portion 71, a proximally located engaging portion 72, and a pivot portion 73 there between. In use, the user would pull or rotate the handle portion 72 about the pivot portion 73 to releasably disengage the complementary teeth 223 of the pawl 222 from the one or more teeth 408 of the elongate member 400.

Various ratchet assemblies and locking mechanism are known the art and may be used in alternate embodiments of the retractor 10. For example, the one or more teeth 408 may be replaced by notches in the central portion 405 of the elongate member 400, or any other feature or profile that provides a surface to abut a partition of a pawl 222 to resist movement of the central portion 405 of the elongate member 400 in one direction. It is also envisaged in other embodiments that the engagement means 220 may include other known releasable locking mechanisms, such as any fastener known in the art and can include permanent and releasable fasteners. For example the engagement means 220 may be a buckle, sap-fit fitting, screw, Velcro, or clamp etc.

The retractor 10 is typically dimensioned such that the length of the elongate member 400 is sufficient to traverse both the working port 21 and the viewing port 31 and the distance between the entrances to both ports of the skin of the patient 95. Typically the depth of the ports used for spinal surgery ranges from 25 mm to 120 mm and the distance between the ports entrances may be from 15-35 mm. Accordingly, the length of the elongate member 400 may vary from 65 mm to 275 mm or more, noting that, when in use, any portion of the tail 200 and elongate member 400 that protrudes beyond the head 300 can be clipped or secured so as to not interfere with the surgical procedure.

The above features of the retractor 10 may be formed from any material that is biocompatible in the short term. The material is preferably sufficiently flexible to allow the retractor 10, in particular the elongate member 400, to conform with the viewing port 20 and the working port 30 and for the tail 300 to meet the head 200 adjacent the skin of the patient 95, when in use.

Accordingly, the features of the retractor 10 are preferably formed from a polymeric material, for example silicone, polyvinylchloride (PVC), polyethylene (PE), polypropylene (PP), polytetrafluoroethylene (PTFE), polymethylmethacrylate (PMMA), trimethylcarbonate, or trimethylene carbonate-lactide, or poly(trimethylene carbonate-co-L-lactide).

In some embodiments, each portion of the retractor 10 may be made from the same or a different material. For example the head 200, tail 300 and/or elongate member 400 may be formed from PVC, while the first flange 409 and/or the second flange 410 may independently be formed from silicone or polypropylene.

The material allows the retractor 10 to traverse a working port 20 and a viewing port 30 such that, in use, the tail 300 is advanced into the head 200 to retract tissue 95 surrounding the working port 20 and the viewing port 30.

There is also provided a method of maintaining triangulation during a surgical procedure, such as biportal endoscopic surgery, and this example will be described with reference to the illustrated embodiment.

Once the working port 20 and viewing port 30 have been established at the respective surgical sites 21, 31 using conventional surgical techniques, with the retractor 10 provided in an open configuration, the tail 300 of the retractor 10 is inserted into the surgical site of either one of the ports, such as the site 31 of the viewing port 30, and advanced until it can be visualised or identified through the surgical site of the other port, such as the surgical site 21 of the working port 20.

At this time, the tail 300 is advanced through the surgical site 21 of the other port 20 and this may occur with the assistance of a suitable surgical tool such as forceps. Once the retractor 10 exits through the surgical site 31 and has traversed the surgical sites 21, 31 of both ports 20, 30, the tail 300 is advanced into head 200 to retract the tissue 95, with the assistance of the flexible transition zone 224.

Once the tissue 95 is retracted to the surgeons satisfaction, the tail 300 and any unwanted portion of the elongate member 400 that protrudes (such as portions of the tail 300 and/or the elongate member 400 that extend past the exit region 214) may be clipped. At this point the entry tube or external shaft of the endoscope or respective ports 20, 30 can be releasably engaged or retained by the flanges 409, 410 laterally extending from the elongate member 400 (eg, through the removable retaining mechanism described above). When the ports 20, 30 are in position, the flanges 409, 410 of the elongate member 400 space apart the sides of the working channel.

In the illustrated embodiment, the pivot portion 413 of the retractor 10 would advantageously facilitate and maintain an acute bend or angle β adjacent or proximal at the respective internal ends 22, 32 of the working and viewing ports 20, 30 for the duration of the surgical procedure. Preferably, the angle β substantially corresponds with the angle α defined by the one or portions of the elongate member 400. The lower surface 202 and/or the one or more flange members 216, 218 of the head 200 can provide a contact surface defined by these features that is configured to distribute a load onto the patient's skin 95 to thereby stabilize the retractor 10 during the surgical procedure.

Once the surgical procedure is complete, the retractor 10 can be removed simply by either severing the elongate member 400 or releasing the locking mechanism 70, and withdrawing the retractor 10 from the surgical sites 21, 31 of the ports 20, 30.

It is appreciated that the examples of the ports 20, 30 are dimensioned to engage with any suitable surgical instrument in the art. Though the examples in the illustrated embodiment were described as a working port 20 and a viewing port 30, the ports can interchange and/or the dimensions of the respective ports can be adjusted in order to accommodate the required instrument for the surgical procedure.

Although a preferred embodiment of the present invention has been described for illustrative purposes, those skilled in the art will appreciate that various modifications, additions and substitutions are possible, without departing from the scope and spirit of the invention as disclosed in the accompanying claims.

Reference Numeral Guide

| Feature | Reference Numeral |
|---|---|
| Retractor | 10 |
| Working port | 20 |
| Site of Working port | 21 |
| End of Working port | 22 |
| Viewing Port | 30 |
| Site of Viewing Port | 31 |
| End of Viewing port | 32 |
| Release Mechanism | 70 |
| Handle Portion | 71 |
| Engaging Portion | 72 |
| Pivot Portion | 73 |
| Patient's Skin | 95 |
| Head | 200 |
| Housing Structure | 201 |
| Upper Surface | 202 |
| Lower Surface | 204 |
| First Side Wall | 206 |
| Second Side Wall | 208 |
| Aperture | 210 |
| Receiving Region | 212 |
| Exit Region | 214 |
| First Flange Member | 216 |
| Second Flange Member | 218 |
| Engagement Means | 220 |
| Pawl | 222 |
| Complementary Pawl Teeth | 223 |
| Flexible Transition Zone | 224 |
| Bend Portion | 226 |
| Tip Port | 228 |
| Second Transition Zone | 230 |
| Tail | 300 |
| Sloped Portion | 302 |
| Elongate Member | 400 |
| First Portion | 402 |
| Second Portion | 404 |
| Central Portion | 405 |
| Upper Surface | 406 |
| Lower Surface | 407 |
| Teeth | 408 |
| First Flange Walls | 409 |
| Second Flange Walls | 410 |
| First Channel | 411 |
| Second Channel | 412 |
| Relief Zone / Pivot Portion | 413 |

1. A retractor to maintain triangulation during biportal endoscopic surgery, the retractor comprising:
a head and a tail connected by an elongate member;
wherein:
the head is adapted to engage with a central portion adjacent the tail;
a first portion of the elongate member is adapted to engage with a surgical instrument; and
the retractor is adapted to traverse a working port and a viewing port such that, in use, the tail is advanced into the head to retract tissue surrounding the working port and the viewing port.

2. The retractor of claim 1, wherein the elongate member includes one or more relief zones.

3. The retractor of claim 2, wherein the elongate member comprises one or more regions adapted to bend or turn about the one or more relief zones.

4. The retractor of claim 2, wherein the one or more relief zones are integrally formed with the elongate member, or provided as a pivotable structure connecting the one or more regions of the elongate member.

5. The retractor of claim 1, wherein, in use, the retractor is adapted to maintain an angle between the working port and the viewing port, wherein the angle substantially corresponds with an angle defined by the one or more relief zones.

6. The retractor of claim 1, wherein the first portion of the elongate member comprises at least one or more flanges configured to engage with the surgical instrument, and wherein the one or more flanges laterally extend from the central portion of the elongate member to form a channel with a first longitudinal axis that substantially aligns with a longitudinal axis defined by the central portion of the elongate member.

7. The retractor of claim 1, wherein the elongate member provides one or more shaft connection means that are adapted to removably retain the surgical instrument received therein, wherein an inner radius of the one or more shaft connection means optionally corresponds with an outer radius of the surgical instrument, and/or wherein the one or more shaft connection means are substantially C-shaped, U-shaped, or have a rectilinear profile.

8. The retractor of claim 1, wherein the surgical instrument is an endoscope or a working scope, and the first portion of the elongate member is adapted to engage with an insertion tube or an external profile of the surgical instrument.

9. The retractor of claim 6, wherein, in use, the channel is positioned at the working port or the viewing port, to protect the tissue surrounding the respective working port or viewing port.

10. The retractor of claim 1, further comprising a flexible transition zone between the elongate member and the head, wherein the transition zone is configured to retract the tissue surrounding the surgical instrument, wherein the transition zone is optionally in the shape of a substantially arcuate hook defined by a bend portion and a tip portion, wherein the head or the elongate member is configured to turn or pivot about the transition zone.

11. The retractor of claim 1, wherein the head comprises a housing structure and one or more flange members laterally extending thereof,
wherein the one or more flange members and/or a surface of the housing structure define a contact surface configured to distribute a load onto the patient's skin to thereby stabilize the retractor, in use.

12. The retractor of claim 11, wherein the one or more flange members are substantially coplanar or non-coplanar with a plane defined by the surface of the housing structure.

13. The retractor of claim 11, wherein the one or more flange members are substantially flat and dome-shaped.

14. The retractor of claim 1, wherein the head comprises an engagement means configured to engage with the tail and/or the elongate member.

15. The retractor of claim 14, wherein the engagement means is provided as a pawl member, wherein the tail and/or the central portion of the elongate member comprise a plurality of teeth configured to engage with the pawl member.

16. The retractor of claim 14, wherein the engagement means further comprises a release mechanism configured to releasably engage the engagement means with the tail and/or the elongate member.

17. A method of maintaining triangulation during biportal endoscopic surgery, the method comprising:
advancing the tail of a retractor of claim 1, through both a working port and a viewing port
advancing the tail into the head of the retractor to retract tissue surrounding the working port and the viewing port; and
engaging one or more portions of the elongate member of the retractor with a surgical instrument.

18. The method of claim 17, wherein the elongate member is configured to releasably retain the surgical instrument, and wherein the head of the retractor optionally provides a contact surface that is configured to distribute a load onto the patient's skin to thereby stabilise the retractor in use.

19. The method of claim 17, further comprising:
maintaining an angle between the working port and the viewing port throughout the surgical procedure, wherein the angle substantially corresponds with an angle defined by the one or more portions of the elongate member.

20. The method of claim 17, further comprising:
engaging the tail and/or the elongate member with the head of the retractor to thereby define a closed configuration.
